(19) United States
(12) Patent Application Publication
(10) Pub. No.: US 2007/0111216 A1
(43) Pub. Date: May 17, 2007

(54) Methods for identifying polymerase inhibitors

(75) Inventors: Jerry Jendrisak, Madison, WI (US); Agnes Radek, Madison, WI (US); Gary Dahl, Madison, WI (US)

Correspondence Address: MEDLEN & CARROLL, LLP, 101 HOWARD STREET, SUITE 350, SAN FRANCISCO, CA, 94105, US

(73) Assignee: Epicentre Technologies, Madison, MA (US)

(21) Appl. No.: 11/235,911

(22) Filed: Sep. 27, 2005

Related U.S. Application Data
(60) Provisional application No. 60/613,462, filed on Sep. 27, 2004.

(51) Int. Cl. C12Q 1/68

(52) U.S. Cl. 435/6

(57) ABSTRACT

The present invention relates to methods and compositions for the identification of enzyme inhibitors. In particular, the present invention relates to the identification of nucleic acid polymerase inhibitors.

### CROSS-REFERENCE TO RELATED APPLICATION

This application claims priority to U.S. Provisional Patent Application Ser. No. 60/613,462 filed Sep. 27, 2004. The entire disclosure of all priority applications is specifically incorporated herein by reference.

### FIELD OF THE INVENTION

The present invention relates to methods and compositions for the identification of enzyme inhibitors. In particular, the present invention relates to the identification of nucleic acid polymerase inhibitors.

### BACKGROUND OF THE INVENTION

Specific enzyme inhibitors find a wide range of applications in the food and agriculture industries, the medical, pharmaceutical and biotechnology industries, etc. These fields in particular provide numerous commercial applications for effective inhibitors of nucleic acid polymerases. Specific nucleic acid polymerase inhibitors are used in agricultural and live stock pest control, in industrial and academic biomedical research and development programs, and in clinical settings. For example, clinicians face an ever-increasing number of pathogens resistant to existing antibiotics.

The available inhibitors of nucleic acid polymerases are very limited. The antibiotic drug rifampicin is believed to selectively inhibit certain bacterial RNA polymerases and α-amanitin is believed to selectively inhibit certain eukaryotic RNA polymerases.

Disclosures that relate to the identification of modulators of nucleic acid polymerase activity include U.S. Pat. No. 5,635,349 entitled “High-Throughput Screening Assay for Inhibitors of Nucleic Acid Polymerases” by LaMarco et al.; PCT Application No. WO 2004/023093 entitled “Target and Method for Inhibition of Bacterial RNA Polymerase” by Ebright; U.S. patent application No. 2004/0110126 entitled “HCV Polymerase Inhibitor Assay” by Kukolj et al.; U.S. patent application No. 2004/0110187 entitled “In vitro Transcription Assay for T Box Antitermination System” by Henkin et al.; PCT Application No. WO 2004/044228 entitled “A Continuous-Read Assay for the Detection of de Novo HCV RNA Polymerase Activity” by Yagi et al.; U.S. patent application Nos. 2004/0054162 and U.S. 2003/0099950 entitled “Molecular Detection Systems Utilizing Reiterative Oligonucleotide Synthesis” by Hanna; PCT Application No. WO 01/38587 entitled “Continuous Time-Resolved Resonance Energy-Transfer Assay for Polynucleic Acid Polymerases” by Furfine et al.; U.S. patent application No. 2004/0072206 entitled “Method for Identifying Modulators of Transcription” by Erringtion et al.; U.S. patent application No. 2004/0048350 entitled “Crystal of Bacterial Core RNA Polymerase with Rifampicin and Methods of Use Thereof” by Darst et al.; U.S. patent application No. 2004/0048283 entitled “Novel Method for Screening Bacterial Transcription Modulators” by Pau et al.; and U.S. Pat. No. 6,350,879 entitled “Benziso-N(L-Histidine Methylester)-Thiazone, Process for the Preparation Thereof, and Use Thereof for RNA Polymerase Inhibition” by Ranganathan et al., all of which are incorporated herein by reference in their entirety.

What is needed are improved methods for identifying and screening polymerase inhibitors.

### SUMMARY OF THE INVENTION

The present invention relates to methods and compositions for the identification of enzyme inhibitors. In particular, the present invention relates to the identification of nucleic acid polymerase inhibitors.

Accordingly, in some embodiments, the present invention provides a method for identifying an inhibitor of a nucleic acid polymerase activity, comprising: providing a single-stranded circular oligonucleotide template; a nucleic acid polymerase; and a plurality of nucleoside triphosphates; incubating the template, the nucleic acid polymerase, and the nucleoside triphosphates in the presence and absence of a candidate inhibitor. In some embodiments, the candidate inhibitor inhibits transcription by the nucleic acid polymerase. In some embodiments, the method further comprises measuring the presence or absence of a polymerization product formed in the presence and absence of the candidate inhibitor. In some embodiments, the method further comprises the step of comparing an amount of the polymerization product formed in the presence and absence of the candidate inhibitor; wherein a decrease in the amount of the polymerization product formed in the presence of the candidate inhibitor compared to the amount of the polymerization product formed in the absence of the candidate inhibitor indicates that the candidate inhibitor is an inhibitor of the nucleic acid polymerase activity. In some preferred embodiments, the method is performed in the absence of a primer. In other embodiments, the method is performed in the presence of a primer. In some preferred embodiments, the nucleotide sequence of the template is devoid of a polymerase promoter sequence. In other embodiments, the nucleotide sequence of the template comprises a polymerase promoter sequence. In some embodiments, the template is DNA or RNA. In some embodiments, the nucleic acid polymerase is a DNA-dependent RNA polymerase, an RNA-dependent RNA polymerase, a primase, a DNA polymerase, or a reverse transcriptase. In some embodiments, the DNA-dependent RNA polymerase is a prokaryotic RNA polymerase. In some embodiments, the prokaryotic RNA polymerase is S. aureus RNA polymerase. In other embodiments, the DNA-dependent RNA polymerase is a eukaryotic RNA polymerase. In some embodiments, the nucleic acid polymerase is a eukaryotic virus polymerase or a prokaryotic virus polymerase. In some embodiments, comparing the amount of the polymerization product formed in the presence and absence of the candidate inhibitor comprises measuring fluorescence generated from a dye that undergoes fluorescence enhancement upon binding to nucleic acids (e.g., RIBOGREEN, SYBR Gold, and SYBR Green I, or SYBR Green II). In some embodiments, the fluorescence is generated in real time. In some embodiments, comparing the amount of the polymerization product formed in the presence and absence of the candidate inhibitor comprises measuring fluorescence generated from a molecular beacon.

In some embodiments, the nucleic acid components (e.g., single-stranded circular oligonucleotide template, primer, etc.) of the methods are generated during the reaction. In other embodiments, they are prepared prior to a reaction and are added to the reaction fully formed.

The present invention further provides a kit for identifying an inhibitor of a nucleic acid polymerase activity, comprising: a single-stranded circular oligonucleotide template; a nucleic acid polymerase; and a reagent for detection of transcription or polymerization from the template. In some embodiments, the kit further comprises a plurality of nucleoside triphosphates. In some embodiments, the kit further comprises at least one inhibitor of the nucleic acid polymerase or a plurality of inhibitors of the nucleic acid polymerase. In some embodiments, the kit further comprises a primer complementary to the template. In some embodiments, the nucleotide sequence of the template is devoid of a polymerase promoter sequence, while in other embodiments, it comprises a polymerase promoter sequence. In some embodiments, the template is DNA or RNA. In some embodiments, the nucleic acid polymerase is selected from the group consisting of a DNA-dependent RNA polymerase, an RNA-dependent RNA polymerase, a primase, a DNA polymerase, and a reverse transcriptase. In some embodiments, the nucleic acid polymerase is a DNA-dependent RNA polymerase, an RNA-dependent RNA polymerase, a primase, a DNA polymerase, or a reverse transcriptase. In some embodiments, the DNA-dependent RNA polymerase is a prokaryotic RNA polymerase. In some embodiments, the prokaryotic RNA polymerase is S. aureus RNA polymerase. In other embodiments, the DNA-dependent RNA polymerase is a eukaryotic RNA polymerase. In some embodiments, the nucleic acid polymerase is a eukaryotic virus polymerase or a prokaryotic virus polymerase. In some embodiments, the reagent comprises a dye that undergoes fluorescence enhancement upon binding to nucleic acids (e.g., the dye is RIBOGREEN, SYBR Gold, SYBR Green I, or SYBER Green II). In some embodiments, the reagent comprises a molecular beacon.

The present invention also provides a method for detecting RNA polymerase activity in a sample suspected of containing an RNA polymerase, comprising: providing a sample suspected of containing an RNA polymerase; a single-stranded circular oligonucleotide DNA template; and a plurality of nucleoside triphosphates; incubating the DNA template, the sample and the nucleoside triphosphates under conditions such that the RNA polymerase, if present, transcribes the DNA template. In some embodiments, the method further comprises the step of measuring the presence or absence of the RNA product. In some preferred embodiments, the method is performed in the absence of a primer. In other embodiments, the method is performed in the presence of a primer. In some preferred embodiments, the nucleotide sequence of the template is devoid of a polymerase promoter sequence. In other embodiments, the nucleotide sequence of the template comprises a polymerase promoter sequence. In some embodiments, the measurement of the presence or absence of the RNA product is a real-time measurement. In other embodiments, the measurement of the presence or absence of the RNA product is an end-point measurement. In some embodiments, the measurement of the presence or absence of the RNA product comprises measuring fluorescence from a dye that undergoes fluorescence enhancement upon binding to nucleic acids (e.g., RIBOGREEN, SYBR Gold, and SYBR Green I, or SYBR Green II). In other embodiments, the measurement of the presence or absence of the RNA product comprises measuring fluorescence from a molecular beacon.

### DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 is a schematic that illustrates an aspect of the invention in which a DNA template circle is incubated with an RNA polymerase.

FIG. 2 shows an example of using one embodiment of the present invention for the identification of inhibitors that are specific for either prokaryotic or eukaryotic RNA polymerases.

FIG. 3 shows an example for the use of the same single-stranded circular DNA template for a broad range of different RNA polymerases.

FIG. 4 shows an example for using a method of the present invention to detect RNA polymerase inhibitors that are specific to a prokaryotic RNA polymerase (RNAP).

FIG. 5 illustrates the results of an example in which a method of the present invention was used to monitor polymerization activity with a molecular beacon (MB).

FIG. 6 shows an example for using one embodiment of the present invention to show inhibition of RNA polymerase activity.

### DEFINITIONS

A variety of terms are used in describing the present invention. In most cases, only terms that are broad and apply to many aspects of the invention are presented in the “Definitions” section. Other terms are defined as presented in describing the specifications and claims in other sections, including, but not limited to, sections entitled “Summary of the Invention,” “Description of the Figures,” “Detailed Description of the Invention,” and “Examples.” If the same terms or similar terms have been used with different meaning by others, including those presented in the section entitled “Background of the Invention” herein above, the terms when used to describe the present invention, shall nevertheless be interpreted to have the meanings presented below and in the sections related to the specification and claims, unless otherwise expressly stated to the contrary.

As used herein in the specification, “a” or “an” may mean one or more. As used herein in the claim(s), when used in conjunction with the word “comprising,” the words “a” or “an” may mean one or more than one. As used herein “another” may mean at least a second or more.

As used herein, the term “enzyme” refers to molecules or molecule aggregates that are responsible for catalyzing chemical and biological reactions. Such molecules are typically proteins, but can also comprise short peptides, RNAs, ribozymes, antibodies, and other molecules. A molecule that catalyzes chemical and biological reactions is referred to as “having enzyme activity” or “having catalytic activity.”

As used herein, the term “nucleic acid polymerase” refers to an enzyme that catalyzes the synthesis of nucleotides into a chain of nucleotides. In some embodiments, nucleic acid polymerases are “primer dependent,” in that they require an oligonucleotide primer for their activity. Preferred nucleic acid polymerases of the present invention are “primer independent,” in that they do not require a primer for their nucleic acid synthesis activity. Nucleic acid polymerases may be DNA (e.g., synthesize DNA) or RNA (e.g., synthesize RNA) polymerases.

A “DNA-dependent DNA polymerase” is an enzyme that synthesizes a complementary DNA (“cDNA”) copy from a DNA template. Examples are DNA polymerase I from E. coli and bacteriophage T7 DNA polymerase. All known DNA-dependent DNA polymerases require a complementary primer to initiate synthesis.

An “RNA-dependent DNA polymerase” or “reverse transcriptase” is an enzyme that can synthesize a complementary DNA copy (“cDNA”) from an RNA template. All known reverse transcriptases also have the ability to make a complementary DNA copy from a DNA template; thus, they are both RNA- and DNA-dependent DNA polymerases.

A “template” is the nucleic acid molecule that is copied by a nucleic acid polymerase. The synthesized copy is complementary to the template. Both RNA and DNA are always synthesized in the 5′-to-3′ direction. A primer is required for both RNA and DNA templates to initiate synthesis by a DNA polymerase.

As used herein a “primer” is an oligonucleotide (oligo), generally with a free 3′-OH group, for which at least the 3′-portion of the oligo is complementary to a portion of the template and which oligo “binds” (or “complexes” or “anneals” or “hybridizes”), by hydrogen bonding and other molecular forces, to the template to give a primer/template complex for initiation of synthesis by a DNA polymerase, and which is extended (i.e., “primer extended”) by the addition of covalently bonded bases linked at its 3′-end which are complementary to the template in the process of DNA synthesis. The result is a primer extension product. Virtually all DNA polymerases (including reverse transcriptases) that are known require complexing of an oligonucleotide to a single-stranded template (“priming”) to initiate DNA synthesis, whereas RNA replication and transcription (copying of RNA from DNA) generally do not require a primer.

Nucleic acid molecules are said to have “5′ ends” and “3′ ends” because mononucleotides are joined in one direction via a phosphodiester linkage to make oligonucleotides, in a manner such that a phosphate on the 5′-carbon of one mononucleotide sugar moiety is joined to an oxygen on the 3′-carbon of the sugar moiety of its neighboring mononucleotide. Therefore, an end of an oligonucleotide referred to as the “5′ end” if its 5′ phosphate is not linked to the oxygen of the 3′-carbon of a mononucleotide sugar moiety and as the “3′ end” if its 3′ oxygen is not linked to a 5′ phosphate of the sugar moiety of a subsequent mononucleotide.

As used herein, the terms “buffer” or “buffering agents” refer to materials that when added to a solution, cause the solution to resist changes in pH. As used herein, the term “solution” refers to an aqueous or non-aqueous mixture. As used herein, the term “buffering solution” refers to a solution containing a buffering agent. As used herein, the term “reaction buffer” refers to a buffering solution in which an enzymatic reaction is performed. As used herein, the term “storage buffer” refers to a buffering solution in which an enzyme is stored.

As used herein, the term “inhibitor of a nucleic acid polymerase,” refers to a natural or synthetic molecule (e.g., small molecule drug) or mimetic that inhibits the nucleic acid synthesis activity of a nucleic acid polymerase. In some embodiments, the inhibition is at least 20% (e.g., at least 50%, 70%, 80%, 90%, 95%, 98%, 99%, 99.5%) of the synthesis activity as compared to the polymerase in the absence of the inhibitor. Assays for analyzing polymerase activity are described herein and are known in the art.

The term “label” as used herein refers to any atom or molecule that can be used to provide a detectable (preferably quantifiable) effect. In some embodiments, labels are attached to a nucleic acid or protein. Labels include but are not limited to dyes; radiolabels such as ³²P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable.

As used herein, the term “dyes that undergo fluorescence enhancement upon binding to nucleic acids” refers to a dye that generates detectable fluorescence in the presence, but not in the absence of nucleic acids. Exemplary dyes include, but are not limited to, SYBRGREEN I, SYBRGREEN II, RIBOGREEN, and SYBR GOLD.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term “nucleic acid molecule” also encompasses nucleic acids that comprise modified internucleotide sugar linkages, such as, but not limited to alpha-thio linkages, which are resistant to cleavage by some nucleases. Further, the term “nucleic acid molecule” also encompasses nucleic acids that contain sugar analogs of ribose or 2-deoxyribose, such as but not limited to 2′-F-, 2′-amino-, 2′-methoxy-, or 2′-azido-2′-deoxyribonucleotides. If the modified nucleic acid is obtained as a product of polymerization or transcription according to a method of the present invention, those with skill in the art will understand that the corresponding modified nucleoside triphosphate (e.g., alpha-thio, 2′-F-, 2′-amino-, 2-methoxy-, or 2′-azido-nucleoside triphosphate) is used in the polymerization or transcription reaction, and that said modified nucleoside triphosphate must be a substrate of the nucleic acid polymerase used.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely related sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reactions that occur within a natural environment.

The terms “test compound”, “candidate compound” and “candidate inhibitor” are used interchangeably herein and refer to any chemical entity, pharmaceutical, drug, and the like that is a candidate for use to treat or prevent a disease, illness, sickness, or disorder of bodily function (e.g., cancer) or that finds use in research or industrial settings.

As used herein, the term “sample” is used in its broadest sense. In one sense, it is meant to include a specimen or culture obtained from any source, as well as biological and environmental samples. Such examples are not however to be construed as limiting the sample types applicable to the present invention.

A “detection method”, “detection” or “measuring the presence or absence of a polymerization product” as used herein is a composition or method for detecting, whether directly or indirectly, the products of nucleic acid polymerization from a method or assay of the invention. The method of detection is not critical. Any appropriate method of detection can be used, such as, but not limited to, radioactive counting or imaging, colorimetry, fluorescence or luminescence. Detection can comprise the use of a probe. Detection can be in real time, or over time for quantitative detection.

As used herein, the term “probe” refers to an oligonucleotide (i.e., a sequence of nucleotides), whether occurring naturally (e.g., as in a purified restriction digest) or produced synthetically, recombinantly or by PCR amplification, which is capable of hybridizing to another oligonucleotide of interest. A probe may be single-stranded or double-stranded (e.g., and rendered single-stranded or partially single-stranded in use). Probes are useful in the detection, identification and isolation of particular gene sequences. It is contemplated that a probe used in the present invention can be labeled with any “reporter molecule,” so that it is detectable in a detection system, including, but not limited to enzyme (i.e., ELISA, as well as enzyme-based histochemical assays), visible, fluorescent, radioactive, and luminescent systems. It is not intended that the present invention be limited to any particular detection system or label. The terms “reporter molecule” and “label” are used herein interchangeably. In addition to probes, primers and deoxynucleoside triphosphates may contain labels; these labels may comprise, but are not limited to, ³²P, ³³P, ³⁵S, enzymes, or visible, luminescent, or fluorescent molecules (e.g., fluorescent dyes).

“Ligation” as used herein refers to the joining of a 5′-phosphorylated end of one nucleic acid molecule with the 3′-hydroxyl end of another nucleic acid molecule by an enzyme called a “ligase,” although in some methods of the invention, the ligation can be effected by another mechanism. With respect to ligation, a region, portion, or sequence that is “adjacent to” or “contiguous to” or “contiguous with” another sequence directly abuts that region, portion, or sequence.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned hereunder are incorporated herein by reference.

The present invention relates to methods and compositions for the identification of enzyme inhibitors. In particular, the present invention relates to the identification of nucleic acid polymerase inhibitors.

In a series of articles and patents, Eric Kool and co-workers disclosed synthesis of DNA or RNA multimers, meaning multiple copies of an oligomer or oligonucleotide joined end to end (i.e., in tandem) by rolling circle replication or rolling circle transcription, respectively, of a circular DNA template molecule. Rolling circle replication uses a primer and a strand-displacing DNA polymerase, such as phi29 DNA polymerase. With respect to rolling circle transcription, it was shown that circular single-stranded DNA (ssDNA) molecules can be efficiently transcribed by phage and bacterial RNA polymerases (Prakash, G. and Kool, E., J. Am. Chem. Soc. 114: 3523-3527, 1992; Daubendiek, S. L. et al., J. Am. Chem. Soc. 117: 7818-7819, 1995; Liu, D. et al., J. Am. Chem. Soc. 118: 1587-1594, 1996; Daubendiek, S. L. and Kool, E. T., Nature Biotechnol., 15: 273-277, 1997; Diegelman, A. M. and Kool, E. T., Nucleic Acids Res., 26: 3235-3241, 1998; Diegelman, A. M. and Kool, E. T., Chem. Biol., 6: 569-576, 1999; Diegelman, A. M. et al., Bio Techniques 25: 754-758, 1998; Frieden, M. et al., Angew. Chem. Int. Ed. Engl. 38: 3654-3657, 1999; Kool, E. T., Acc. Chem. Res., 31: 502-510, 1998; U.S. Pat. Nos. 5,426,180; 5,674,683; 5,714,320; 5,683,874; 5,872,105; 6,077,668; 6,096,880; and 6,368,802; and U.S. patent application No. 2003/0087241).

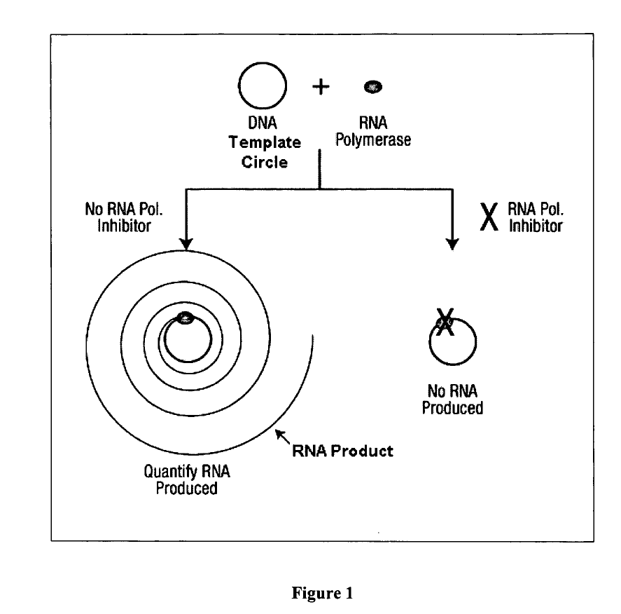

In some embodiments, the present invention provides methods of identifying inhibitors of nucleic acid (e.g., RNA or DNA) polymerases. In preferred embodiments, the methods of the present invention comprise incubating a nucleic acid polymerase with a circular template, nucleotide triphosphates (e.g., NTPs or dNTPs), and the candidate inhibitor. In preferred embodiments, reactions are primer independent (e.g., it is not necessary to include a primer in the reaction mixture). The present invention, however, is not limited to primer independent methods. One exemplary embodiment of the present invention that utilizes a primer-independent reaction on a circular template is shown in FIG. 1.

In one exemplary embodiment, the methods of the present invention are used to screen for candidate inhibitors that inhibit the activity of a nucleic acid polymerase of a pathogenic microorganism (e.g., virus or bacteria) but not a host (e.g., eukaryotic) nucleic acid polymerase. An exemplary method for such an application is described in Example 5.

The present invention is not limited to a particular nucleic acid polymerase. The methods of the present invention are suitable for use with DNA and RNA polymerases. The methods of the present invention are also suitable for use with nucleic acid polymerases derived from a variety of macro and microorganisms including, but not limited to, bacteria (e.g., pathogenic or non pathogenic bacteria), viruses (e.g., prokaryotic or eukaryotic viruses, including pathogenic viruses), eukaryotes (e.g., fungi, plants and animals). The methods of the present invention are illustrated below (See e.g., Experimental Section) with a variety of exemplary, non-limiting nucleic acid polymerases. One skilled in the art recognizes that any polymerase may be used in the methods of the present invention. The present invention is not limited to the analysis of any particular type of virus polymerase or inhibitor. Indeed, the present invention contemplates the analysis of a variety of viruses, including but not limited to, viruses from the following families: Adenoviridae, Arenaviridae, Astroviridae, Bimaviridae, Bunyaviridae, Caliciviridae, Circoviridae, Coronaviridae, Filoviridae, Flaviviridae, Hepadnaviridae, Herpesviridae, Iridoviridae, Filoviridae, Orthomyxoviridae, Papovaviridae, Paramyxoviridae, Parvoviridae, Picornaviridae, Poxyiridae, Reoviridae, Retroviridae, Rhabdoviridae, Togaviridae, Badnavirus, Bromoviridae, Comoviridae, Geminiviridae, Partitiviridae, Potyviridae, Sequiviridae, and Tombusviridae; the following genera: Mastadenovirus, Aviadenovirus, African swine fever-like viruses, Arenavirus, Arterivirus, Astrovirus, Aquabirnavirus, Avibimavirus, Bunyavirus, Hantavirus, Nairovirus, Phlebovirus, Calicivirus, Circovirus, Coronavirus, Torovirus, Deltavirus, Filovirus, Flavivirus, Japanese Encephalitis Virus group, Pestivirus, Hepatitis C—like viruses, Orthohepadnavirus, Avihepadnavirus, Simplexvirus, Varicellovirus, Cytomegalovirus, Muromegalovirus, Roseolovirus, Lymphocryptovirus, Rhadinovirus, Ranavirus, Lymphocystivirus, Goldfish virus-like viruses, Influenzavirus A, B, Influenzavirus C, Thogoto-Like viruses, Polyomavirus, Papillomavirus, Paramyxovirus, Morbillivirus, Rubulavirus, Pneumovirus, Parvovirus, Erythrovirus, Dependovirus, Enterovirus, Rhinovirus, Hepatovirus, Cardiovirus, Aphthovirus, Orthopoxvirus, Parapoxvirus, Avipoxvirus, Capripoxvirus, Leporipoxvirus, Suipoxvirus, Molluscipoxvirus, Yatapoxvirus, Orthoreovirus, Orbivirus, Rotavirus, Coltivirus, Aquareovirus, mammalian type B retroviruses, mammalian type C retroviruses, avian type C retroviruses, type D retroviruses, blv-htlv retroviruses, Lentivirus, Spumavirus, Vesiculovirus, Lyssavirus, Ephemerovirus, Alphavirus, Rubivirus, Badnavirus, Alfamovirus, Ilarvirus, Bromovirus, Cucumovirus, Tospovirus, Capillovirus, Carlavirus, Caulimovirus, Closterovirus, Comovirus, Fabavirus, Nepovirus, Dianthovirus, Enamovirus, Furovirus, Subgroup I Geminivirus, Subgroup II Geminivirus, Subgroup III Geminivirus, Hordeivirus, Idaeovirus, Luteovirus, Machlomovirus, Marafivirus, Necrovirus, Partitiviridae, Alphacryptovirus, Betacryptovirus, Potexvirus, Potyvirus, Rymovirus, Bymovirus, Fijivirus, Phytoreovirus, Oryzavirus, Nucleorhabdovirus, Sequivirus, Waikavirus, Sobemovirus, Tenuivirus, Tobamovirus, Tobravirus, Carmovirus, Tombusvirus, Trichovirus, Tymovirus, Umbravirus; and the following species: human adenovirus 2, fowl adenovirus 1, African swine fever virus, lymphocytic choriomeningitis virus, equine arteritis virus, human astrovirus 1, infectious pancreatic necrosis virus, infectious bursal disease virus, Bunyamwera virus, Hantaan virus, Nairobi sheep disease virus, sandfly fever Sicilian virus, vesicular exanthema of swine virus, chicken anemia virus, avian infectious bronchitis virus, Berne virus, hepatitis delta virus, Marburg virus, yellow fever virus, west Nile virus, bovine diarrhea virus, hepatitis C virus, hepatitis B virus, duck hepatitis B virus, human herpesvirus 1, human herpesvirus 3, human herpesvirus 5, human cytomegalovirus, mouse cytomegalovirus 1, human herpesvirus 6, human herpesvirus 4, ateline herpesvirus 2, frog virus 3, flounder virus, goldfish virus 1, influenza A virus, influenza B virus, influenza C virus, Thogoto virus, murine polyomavirus, cottontail rabbit papillomavirus (Shope), Paramyxovirus, human parainfluenza virus 1, measles virus, mumps virus, human respiratory syncytial virus, mice minute virus, B 19 virus, adeno-associated virus 2, poliovirus 1, human rhinovirus 1A, porcine rhinovirus, hepatitis A virus, encephalomyocarditis virus, St. Louis encephalomyocarditis virus, foot-and-mouth disease virus O, vaccinia virus, orf virus, fowlpox virus, sheeppox virus, monkey pox virus, myxoma virus, swinepox virus, Molluscum contagiosum virus, Yaba monkey tumor virus, reovirus 3, bluetongue virus 1, simian rotavirus SA11, Colorado tick fever virus, golden shiner virus, mouse mammary tumor virus, murine leukemia virus, avian leukosis virus, Mason-Pfizer monkey virus, bovine leukemia virus, human immunodeficiency virus 1, human spumavirus, vesicular stomatitis Indiana virus, rabies virus, bovine ephemeral fever virus, Sindbis virus, rubella virus, commelina yellow mottle virus, alfalfa mosaic virus, tobacco streak virus, brome mosaic virus, cucumber mosaic virus, tomato spotted wilt virus, apple stem grooving virus, carnation latent virus, cauliflower mosaic virus, beet yellows virus, cowpea mosaic virus, broad bean wilt virus 1, tobacco ringspot virus, carnation ringspot virus, pea enation mosaic virus, soil-borne wheat mosaic virus, maize streak virus, beet curly top virus, bean golden mosaic virus, barley stripe mosaic virus, raspberry bushy dwarf virus, barley yellow dwarf virus, maize chlorotic mottle virus, maize rayado fino virus, tobacco necrosis virus, white clover cryptic virus 1, white clover cryptic virus 2, potato virus X, potato virus Y, ryegrass mosaic virus, barley yellow mosaic virus, Fiji disease virus, wound tumor virus, rice ragged stunt virus, potato yellow dwarf virus, tobacco necrosis satellite, parsnip yellow fleck virus, rice tungro spherical virus, Southern bean mosaic virus, rice stripe virus, tobacco mosaic virus, tobacco rattle virus, carnation mottle virus, tomato bushy stunt virus, apple chlorotic leaf spot virus, turnip yellow mosaic virus, and carrot mottle virus.

The present invention is not limited to the analysis of any particular type of bacteria polymerase. Indeed, the analysis of variety of bacteria is contemplated, including, but not limited to, Gram-positive cocci such as Staphylococcus aureus, Streptococcus pyogenes (group A), Streptococcus spp. (viridans group), Streptococcus agalactiae (group B), S. bovis, Streptococcus (anaerobic species), Streptococcus pneumoniae, and Enterococcus spp.; Gram-negative cocci such as Neisseria gonorrhoeae, Neisseria meningitidis, and Branhamella catarrhalis; Gram-positive bacilli such as Bacillus anthracis, Bacillus subtilis, Corynebacterium diphtheriae and Corynebacterium species which are diptheroids (aerobic and anerobic), Listeria monocytogenes, Clostridium tetani, Clostridium difficile, Escherichia coli, Enterobacter species, Proteus mirablis and other spp., Pseudomonas aeruginosa, Klebsiella pneumoniae, Campylobacter jejuni, Legionella peomophilia, Mycobacterium tuberculosis, Clostridium tetani, Hemophilus influenzae, Neisseria gonorrhoeae, Treponema pallidum, Bacillus anthracis, Vibrio cholerae, Borrelia burgdorferi, Cornebacterium diphtheria, Bacillus anthracis, and other members of the following genera: Vibrio, Salmonella, Shigella, Pseudomonas, Actinomyces, Aeromonas, Bacillus, Bacteroides, Bordetella, Brucella, Campylobacter, Capnbocylophaga, Chlamydia, Clostridium, Corynebacterium, Eikenella, Erysipelothriz, Escherichia, Fusobacterium, Hemophilus, Klebsiella, Legionella, Leptospira, Listeria, Mycobacterium, Mycoplasma, Neisseria, Nocardia, Pasteurella, Proteus, Pseudomonas, Rickettsia, Salmonella, Selenomonas, Shigelia, Staphylococcus, Streptococcus, Treponema, Bibro, and Yersinia.

The present invention can also be used for analyzing the activity of RNA polymerases of fungi of any type, or for assaying for inhibitors of RNA polymerases of fungi of any type, including but not limited to yeast or other fungi that are pathogenic or beneficial for humans, plants or animals.

Candidate Inhibitors

The present invention is also not limited to a particular candidate inhibitor. A variety of commercial sources and methods of generating test compounds are known in the art. The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

Candidate inhibitors of the invention can be nucleic acids. “SELEX,” as described by Gold and Tuerk in U.S. Pat. No. 5,270,163, can be used to select a nucleic acid for use as an inhibitor according to the invention. SELEX permits selection of a nucleic acid molecule that has high affinity for a specific analyte from a large population nucleic acid molecules, at least a portion of which have a randomized sequence. For example, a population of all possible randomized 25-mer oligonucleotides (i.e., having each of four possible nucleic acid bases at every position) will contain 4²⁵ (or 10¹⁵) different nucleic acid molecules, each of which has a different three-dimensional structure and different analyte binding properties. SELEX can be used, according to the methods described in U.S. Pat. Nos. 5,270,163; 5,567,588; 5,580,737; 5,587,468; 5,683,867; 5,696,249; 5723,594; 5,773,598; 5,817,785; 5,861,254; 5,958,691; 5,998,142; 6,001,577; 6,013,443; 6,030,776; and 6,300,074, incorporated herein by reference, in order to select an analyte-binding nucleic acid with high affinity for the restriction activity domain of the R-M system of the host cell. A polynucleotide or oligonucleotide inhibitor of the invention that is obtained using SELEX may comprise naturally occurring nucleic acid bases, sugar moieties, or internucleoside linkages or one or more non-naturally occurring nucleic acid bases, sugar moieties, or internucleoside linkages.

Circular Templates

A circular oligonucleotide template can be prepared from a linear precursor, i.e., a linear precircle. The linear precircle preferably has a 3′- or 5′-phosphate group and can contain any desired DNA or RNA or analog thereof. Preferably, a circular template has about 15-1500 nucleotides, more preferably about 24-500, and most preferably about 30-150 nucleotides, although other lengths are contemplated.

Linear precircle oligonucleotides, from which the circular template oligonucleotides are prepared, can be made by any of a variety of procedures known for making DNA and RNA oligonucleotides. For example, the linear precircle can be synthesized by any of a variety of known techniques, such as enzymatic or chemical, including automated synthetic methods. Furthermore, the linear oligomers used as the template linear precircle can be synthesized using rolling circle methods. Many linear oligonucleotides are available commercially, and can be phosphorylated on either end by any of a variety of techniques. Linear precircle oligonucleotides can also be restriction endonuclease fragments derived from naturally occurring DNA sequence. Briefly, DNA isolated from an organism can be digested with one or more restriction enzymes. The desired oligonucleotide sequence can be isolated and identified by standard methods as described in Sambrook et al., A Laboratory Guide to Molecular Cloning, Cold Spring Harbor, N.Y. (1989). The desired oligonucleotide sequence can contain a cleavable site, or a cleavable site can be added to the sequence by ligation to a synthetic linker sequence by standard methods.

Linear precircle oligonucleotides can be purified by polyacrylamide gel electrophoresis, or by any number of chromatographic methods, including gel filtration chromatography and high performance liquid chromatography. To confirm a nucleotide sequence, oligonucleotides can be subjected to RNA or DNA sequencing by any of the known procedures. This includes Maxam-Gilbert sequencing, Sanger sequencing, capillary electrophoresis sequencing, automated sequencing, wandering spot sequencing procedure, or by using selective chemical degradation of oligonucleotides bound to Hybond paper. Sequences of short oligonucleotides can also be analyzed by plasma desorption mass spectroscopy or by fast atom bombardment.

The present invention also provides several methods wherein the linear precircles are then ligated chemically or enzymatically into circular form. This can be done using any standard techniques that result in the joining of two ends of the precircle. Such methods include, for example, chemical methods employing known coupling agents such as BrCN plus imidazole and a divalent metal, N-cyanoimidazole with ZnCl₂, 1-(3-dimethylaminopropyl)-3 ethylcarbodiimide HCl, and other carbodiimides and carbonyl diimidazoles. Furthermore, the ends of a precircle can be joined by condensing a 5′-phosphate and a 3′-hydroxyl, or a 5′-hydroxyl and a 3′-phosphate. Enzymatic circle closure is also possible using a ligase under appropriate reaction conditions.

The invention is not limited to a specific ligase for circularizing a linear precircle and different ligases and ligation methods can be used in different embodiments of the invention. Since intramolecular ligation is generally much more efficient than intermolecular ligation, THERMOPHAGE RNA Ligase (Prokaria Ltd., Reykjavik, Iceland), an enzyme derived from the thermophilic phage RM378 that infects thermophilic eubacterium Rhodothermus marinus and that ligates the 5′-phosphate and 3′-hydroxyl termini of single-stranded DNA or RNA, is a preferred ligase for circularizing a linear precircle in some embodiments of the invention. In some other embodiments, CIRCLIGASE ssDNA Ligase (EPICENTRE Biotechnologies, Madison, Wis., USA) or THERMOPHAGE ssDNA ligase (Prokaria Ltd., Reykjavik, Iceland), which is encoded by the thermophilic phage TS2126 that infects Thermus scotoductus (U.S. Pat. No. 6,492,161), is used for circularizing a linear precircle.

RNA Ligase can also ligate single-stranded DNA or RNA molecules. Faruqui discloses in U.S. Pat. No. 6,368,801, incorporated herein by reference, that T4 RNA ligase can efficiently ligate DNA ends of nucleic acids that are adjacent to each other when hybridized to an RNA strand. A ligation splint can improve the specificity of ligation in some applications. A “ligation splint oligo” or “ligation splint” is an oligo that is used to provide an annealing site or a “ligation template” for joining two ends of one nucleic acid (i.e., “intramolecular joining”) or two ends of two nucleic acids (i.e., “intermolecular joining”) using a ligase or another enzyme with ligase activity. The ligation splint holds the ends adjacent to each other and “creates a ligation junction” between the 5′-phosphorylated and a 3′-hydroxylated ends that are to be ligated.

Thus, T4 RNA ligase is a preferred ligase of the invention in embodiments in which DNA ends are ligated on a ligation splint oligo comprising RNA. Ligation splints comprising RNA can be removed by digestion with RNase H following ligation, which is an advantage in some embodiments. T4 DNA ligase, an ATP-dependent ligase, or an NAD-dependent ligase, such as but not limited to E. coli DNA ligase, Tth DNA ligase, Tfl DNA ligase, or AMPLIGASE DNA Ligase (EPICENTRE Biotechnologies, Madison, Wis., USA) can be used in some embodiments of the invention, in which case a DNA ligation splint oligo can be used; the ligation splint oligo and unligated linear precircles can then be removed by digestion with a single-strand-specific exonuclease, such as exonuclease I (which can be inactivated by heat treatment). The invention is also not limited to the use of a ligase for enzymatically joining the 5′-end to the 3′-end of the same or different nucleic acid molecules in the various embodiments of the invention. By way of example, other enzymatic ligation methods such as, but not limited to, topoisomerase-mediated ligation (e.g., U.S. Pat. No. 5,766,891, incorporated herein by reference) can be used.

The ends of the linear oligonucleotide precircle can alternatively be joined using a self-ligation reaction. In this method, the 5′ end of the linear precircle is 5′-iodo- or 5′-tosyl- and the 3′ end is 3′-phosphorothioate.

The circular oligonucleotide template can be purified by standard techniques although this may be unnecessary. For example, if desired the circular oligonucleotide template can be separated from the end-joining group by denaturing gel electrophoresis or melting followed by gel electrophoresis, size selective chromatography, or other appropriate chromatographic or electrophoretic methods. Also, linear DNA or RNA molecules can be removed from the circular oligonucleotide template by digestion with an exonuclease or exoribonuclease, respectively. Preferably, the exonuclease or exoribonuclease can be inactivated by heat treatment. For example, but without limitation, exonuclease I can be used to digest linear DNA molecules and TERMINATOR Exonuclease (EPICENTRE Biotechnologies, Madison, Wis., USA) can be used to digest linear single-stranded RNA (or DNA) having a 5′-phosphate group. The isolated circular oligonucleotide can be further purified by standard techniques as needed.

A variety of methods are known in the art for making nucleic acids having a particular sequence or that contain particular nucleic acid bases, sugars, internucleoside linkages, chemical moieties, and other compositions and characteristics. Any one or any combination of these methods can be used to make a nucleic acid, polynucleotide, or oligonucleotide for the present invention. Said methods include, but are not limited to: (1) chemical synthesis (usually, but not always, using a nucleic acid synthesizer instrument); (2) post-synthesis chemical modification or derivatization; (3) cloning of a naturally occurring or synthetic nucleic acid in a nucleic acid cloning vector (e.g., see Sambrook, et al., Molecular Cloning: A Laboratory Approach 2^{nd} ed., Cold Spring Harbor Laboratory Press, 1989) such as, but not limited to a plasmid, bacteriophage (e.g., m13 or lamda), phagemid, cosmid, fosmid, YAC, or BAC cloning vector, including vectors for producing single-stranded DNA; (4) primer extension using an enzyme with DNA template-dependent DNA polymerase activity, such as, but not limited to, Klenow, T4, T7, rBst, Taq, Tfl, or Tth DNA polymerases, including mutated, truncated (e.g., exo-minus), or chemically-modified forms of such enzymes; (5) PCR (e.g., see Dieffenbach, C. W., and Dveksler, eds., PCR Primer: A Laboratory Manual, 1995, Cold Spring Harbor Laboratory Press, Cold Spring Harbor, N.Y.); (6) reverse transcription (including both isothermal synthesis and RT-PCR) using an enzyme with reverse transcriptase activity, such as, but not limited to, reverse transcriptases derived from avian myeloblasosis virus (AMV), Maloney murine leukemia virus (MMLV), Bacillus stearothermophilus (rBst), Thermus thermophilus (Tth); (7) in vitro transcription using an enzyme with RNA polymerase activity, such as, but not limited to, SP6, T3, or T7 RNA polymerase, Tth RNA polymerase, E. coli RNA polymerase, or another enzyme; (8) use of restriction enzymes and/or modifying enzymes, including, but not limited to exo- or endonucleases, kinases, ligases, phosphatases, methylases, glycosylases, terminal transferases, including kits containing such modifying enzymes and other reagents for making particular modifications in nucleic acids; (9) use of polynucleotide phosphorylases to make new randomized nucleic acids; (10) other compositions, such as, but not limited to, a ribozyme ligase to join RNA molecules; and/or (11) any combination of any of the above or other techniques known in the art. Oligonucleotides and polynucleotides, including chimeric (i.e., composite) molecules and oligonucleotides with non-naturally-occurring bases, sugars, and internucleoside linkages are commercially available (e.g., see the 2000 Product and Service Catalog, TriLink Biotechnologies, San Diego, Calif., USA; www.trilinkbiotech.com)

Nucleic Acid Labels

Nucleic acid polymerization products may be labeled with any art-known detectable marker, including radioactive labels such as ³²P, ³⁵S, ³H, and the like; fluorophores; chemiluminescers; or enzymatic markers, with fluorescent labels preferred such as fluorescein isothiocyanate, lissamine, Cy3, Cy5, and rhodamine 110, with Cy3 and Cy5 particularly preferred. Suitable fluorophore moieties that can be selected as labels include, but are not limited to, 4-acetamido-4′-isothiocyanatostilbene-2,2′disulfonic acid acridine and derivatives: acridines, acridine isothiocyanate, 5-(2′-aminoethyl)aminona-phthalene-1-sulfonic acid (EDANS), 4-amino-N-[3-vinylsulfonyl)phenyl]napht-halimide-3,5 disulfonate (Lucifer Yellow VS), -(4-anilino-1-naphthyl)malei-mide, anthranilimide, Brilliant Yellow, coumarin and derivatives: coumarin, 7-amino-4-methylcoumarin (AMC, Coumarin 120), 7-amino-4-trifluoromethylcoumarin (Coumarin 151), Cy3, Cy5, cyanosine, 4′,6-diaminidino-2-phenylindole (DAPI), 5′,5″-dibromopyrogallol-sulfoneph-thalein (Bromopyrogallol Red), 7-diethylamino-3-(4′-isothiocyanatophenyl)-4-methylcoumarin, diethylenetriamine pentaacetate, 4,4′-diisothiocyanatodi-hydro-stilbene-2,2′-disulfonic acid, 4,4′-diisothiocyanatostilbene-2,2′-di-sulfonic acid, 5-[dimethylamino]naphthalene-1-sulfonyl chloride (DNS, dansyl chloride), 4-(4′-dimethylaminophenylazo)benzoic acid (DABCYL), 4-dimethylaminophenylazophenyl-4′-isothiocyanate (DABITC), eosin and derivatives: eosin, eosin isothiocyanate, erythrosin and derivatives: erythrosin B, erythrosin isothiocyanate, ethidium, fluorescein and derivatives: 5-carboxyfluorescein (FAM), 5-(4,6-dichlorotriazin-2-yl)amin-ofluorescein (DTAF), 2′7′-dimethoxy-4′5′-dichloro-6-carboxyfluorescein (JOE), fluorescein, fluorescein isothiocyanate, QFITC (XR1TC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene and derivatives: pyrene, pyrene butyrate, succinimidyl 1-pyrene butyrate, Reactive Red 4 (Cibacron® Brilliant Red 3B-A), rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 110, rhodamine 123, rhodamine X isothiocyanate, sulforhodamine B, sulforhodamine 101, sulfonyl chloride derivative of sulforhodamine 101 (Texas Red), N,N,N′N′-tetramethyl-6-carboxyrhodamine (TAMRA), tetramethyl rhodamines, tetramethyl rhodamine isothiocyanate (TRITC), riboflavin, rosolic acid, terbium chelate derivatives.

Not only fluorophores, but also chemiluminescers and enzymes, among others, may be used as labels. In yet another embodiment, the polymerization products may be labeled with an enzymatic marker that produces a detectable signal when a particular chemical reaction is conducted, such as alkaline phosphatase or horseradish peroxidase. Such enzymatic markers are preferably heat stable, so as to survive the second strand synthesis and denaturing steps of the amplification process of the present invention.

Kits

The invention also comprises kits and compositions (e.g., reaction mixtures, etc.) for a method of the invention. A kit is a combination of individual compositions useful or sufficient for carrying out one or more steps a method of the invention, wherein the compositions are optimized for use together in the method. A composition comprises an individual component for at least one step of a method of the invention. The present invention further provides a kit for identifying an inhibitor of a nucleic acid polymerase activity, comprising: a single-stranded circular oligonucleotide template; a nucleic acid polymerase; and a reagent for detection of transcription from the template. In some embodiments, the kit further comprises a plurality of nucleoside triphosphates. In some embodiments, the kit further comprises a plurality of inhibitors of the nucleic acid polymerase. In some embodiments, the kit further comprises a primer complementary to the template. In some embodiments, the nucleotide sequence of the template is devoid of a polymerase promoter sequence, while in other embodiments, it comprises a polymerase promoter sequence. In some embodiments, the template is DNA or RNA. In some embodiments, the nucleic acid polymerase is selected from the group consisting of a DNA-dependent RNA polymerase, an RNA-dependent RNA polymerase, a primase, a DNA polymerase, and a reverse transcriptase. In some embodiments, the nucleic acid polymerase is a DNA-dependent RNA polymerase, an RNA-dependent RNA polymerase, a primase, a DNA polymerase, or a reverse transcriptase. In some embodiments, the DNA-dependent RNA polymerase is a prokaryotic RNA polymerase. In some embodiments, the prokaryotic RNA polymerase is S. aureus RNA polymerase. In other embodiments, the DNA-dependent RNA polymerase is a eukaryotic RNA polymerase. In some embodiments, the nucleic acid polymerase is a eukaryotic virus polymerase or a prokaryotic virus polymerase. In some embodiments, the reagent comprises a dye that undergoes fluorescence enhancement upon binding to nucleic acids (e.g., the dye is RIBOGREEN, SYBR Gold, SYBR Green I, or SYBER Green II). In some embodiments, the reagent comprises a molecular beacon. In some embodiments, the kit further comprises control reagents (e.g., sample polymerases and/or inhibitors for positive controls, polymerase and/or inhibitor minus samples for negative controls, etc.). In some embodiments, the kit further comprises instructions for carryout out the methods. In some embodiments, the instructions are embodied in computer software that assists the user in obtaining, analyzing, displaying, and/or storing results of the method. The software may further comprise instructions for managing sample information, integrating with scientific equipment (e.g., detection equipment), etc.

Methods of Detecting Polymerase Activity

The present invention also provides a method for detecting RNA polymerase activity in a sample suspected of containing an RNA polymerase, comprising: providing a sample suspected of containing an RNA polymerase; single-stranded circular oligonucleotide DNA template; and a plurality of nucleoside triphosphates; incubating the DNA template, the sample and the nucleoside triphosphates under conditions such that the RNA polymerase, if present, transcribes the DNA template. In some embodiments, the method further comprises the step of measuring the presence or absence of the RNA product. In some preferred embodiments, the method is performed in the absence of a primer. In other embodiments, the method is performed in the presence of a primer. In some preferred embodiments, the nucleotide sequence of the template is devoid of a polymerase promoter sequence. In other embodiments, the nucleotide sequence of the template comprises a polymerase promoter sequence. In some embodiments, the measurement of the presence or absence of the RNA product is a real-time measurement. In other embodiments, the measurement of the presence or absence of the RNA product is an end-point measurement. In some embodiments, the measurement of the presence or absence of the RNA product comprises measuring fluorescence from a dye that undergoes fluorescence enhancement upon binding to nucleic acids (e.g., RIBOGREEN, SYBR Gold, and SYBR Green 1, or SYBR Green II). In other embodiments, the measurement of the presence or absence of the RNA product comprises measuring fluorescence from a molecular beacon.

All numerical ranges in this specification are intended to be inclusive of their upper and lower limits.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Rolling Circle Transcription with Different RNA Polymerases and Subsequent Detection of Transcription Products Without Sample Cleanup

A single-stranded circularized 45mer DNA molecule was obtained having the following sequence:

(Sequence I.D. No. 1)

CTGGAGGAGATTTTGTGGTATCGATTCGTCTCTTAGAGGAAGCTA.

A reaction mixture was prepared containing 30 ng (about 2 pmoles) of the circularized 45mer, 50 units (about 1 pmole) of T7 RNA polymerase, T3 RNA polymerase, or SP6 RNA polymerase (available from EPICENTRE Biotechnologies, Madison, Wis.), 28 units of RNASIN Plus RNase Inhibitor (available from Promega, Madison, Wis.), and a reaction buffer. The reaction mixture was incubated at 37° C. for 5 minutes. Subsequently, a transcription reaction was started by adding ATP, CTP, GTP and UTPs such that the final concentration of each NTP was 1 mM. The final reaction mixture had a volume of 25 μl and was incubated for 3 hours at 37° C. The transcription reaction was stopped by adding 14 mM EDTA.

An aliquot of the transcription reaction mixture was analyzed by agarose gel electrophoresis. To determine the quantity of the RNA, another aliquot of the transcription reaction mixture was added to 2 ml RIBOGREEN reagent (Molecular Probes), which had been diluted 1:2,000 in 10 mM Tris-1 mM EDTA pH 8.0. Subsequently, fluorescence was measured on a TURNER QUANTECH fluorometer (Barnstead/Thermolyne) and compared to a standard curve prepared with a known amount of RNA.

The results indicated that RNA was synthesized with all three RNA polymerases used. The reaction with T7 RNA polymerase yielded about 1,000 ng RNA, the reaction with T3 RNA polymerase yielded about 210 ng RNA and the reaction with SP6 RNA polymerase yielded about 170 ng RNA. The results further indicate that a sample cleanup was not necessary before adding the RIBOGREEN reagent and measuring the fluorescent emission of the RIBOGREEN bound to the transcription product illustrating the fact that the use of single-stranded circular templates results in a low background.

### Example 2

### Circular and Linear Single-Stranded DNA Molecules as Templates for Transcription

A single-stranded circularized 38mer DNA molecule and a single-stranded linear 38mer molecule were obtained, both having the following sequence:

(Sequence I.D. No. 2)

CAAAAGAAGCGGAGCTTCTTUTTTTTTTTTTTTTTTTT.

A reaction mixture was prepared containing 14 pmoles of the single-stranded circularized 38mer DNA molecule or the single-stranded linear 38 mer molecule, AMPLISCRIBE T7 Enzyme Solution containing T7 RNA polymerase (EPICENTRE), 7.5 mM of each NTP, 2 μg Single-Strand DNA Binding Protein (EPICENTRE), 10 mM DTT, and AMPLISCRIBE T7 Buffer (EPICENTRE) in a volume of 22 μl. The reaction mixture was incubated for 2 hours at 37° C. and the reaction products were visualized on a denaturating formaldehyde-agarose gel.

The results show that RNA was only synthesized from the circularized template, whereas the reaction with the linear template did not yield any detectable RNA products. This finding demonstrated that a circular single-stranded template and not a linear template can be used for transcription reactions performed in the absence of a promoter sequence or a primer.

### Example 3

### Inhibition of the Activity of Different RNA Polymerases Detected by Real-Time Detection and Gel Electrophoresis

A single-stranded circularized 81 mer DNA molecule was obtained having the following sequence:

(Sequence I.D. No. 3)

AGTCCTCAGTCCACGTGGTTTTTTTTTTTTTTTTTTTTTTTGCGCTAGGG
ATAACAGGGTAATCATTGCCGTCTGAAGAGG.

A reaction mixture was prepared containing 0.25 units of E. coli RNA Polymerase Core Enzyme, E. coli RNA polymerase Holoenzyme (both RNA polymerases are available from EPICENTRE), or S. aureus RNA polymerase, and a reaction buffer. The reaction mixture was incubated in the presence or absence of 10 μM rifampicin for 10 min at 37° C. Subsequently, 0.8 pmole of the circularized 81 mer DNA oligomer were added to the reaction mixture followed by incubation for 5 min at 37° C. Then SYBR Gold (Molecular Probes), which had been diluted 1:20,000, was added. Subsequently, ATP, CTP, GTP and UTP were added such that the concentration of each NTP was 1 mM in the final reaction mixture.

Fluorescence was measured for about 3 hours in an iCycler iQ real-time PCR detection system (Bio-Rad Laboratories) using 490 nm excitation and 530 nm emission wavelengths. Afterwards, the reaction products were analyzed by gel electrophoresis.

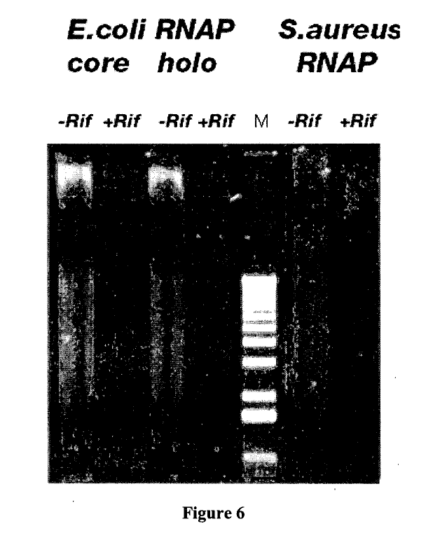

The results indicated that RNA was synthesized in the absence of rifampicin, whereas no RNA was synthesized in the presence of rifampicin by E. coli RNA Polymerase Core Enzyme, E. coli RNA polymerase Holoenzyme or S. aureus RNA polymerase (FIG. 6). These results illustrate that the methods of the present invention can be used show inhibition of RNA polymerase activity by rifampicin. The results also show that rolling circle transcription can be performed in the presence of a fluorescent dye.

### Example 4

### Inhibition of the Activity of a Bacterial RNA Polymerase Monitored in Real-Time

A single-stranded circularized 45mer DNA molecule having the same sequence as the circular 45mer used in Example 1 (Sequence I.D. No. 1) was obtained.

A reaction mixture was prepared containing 0.5 units of E. coli RNA Polymerase Core Enzyme, and a reaction buffer. The reaction mixture was incubated in the presence of 1 μM rifampicin, 20 U TAGETIN (Epicentre Biotechnologies), or 20 μg/ml α-amanitin for 10 min at 37° C. Subsequently, 0.8 pmole of the circularized 45mer DNA oligomer were added to the reaction mixture followed by incubation for 5 min at 37° C. Then SYBR Gold (Molecular Probes), which had been diluted 1:20,000, was added. Subsequently, ATP, CTP, GTP and UTP were added such that the concentration of each NTP was 0.5 mM in the final reaction mixture. Fluorescence was measured for about 1.5 hours in an iCycler iQ real-time PCR detection system (Bio-Rad Laboratories) using 490 nm excitation and 530 nm emission wavelengths. Afterwards, the reaction products were analyzed by gel electrophoresis.

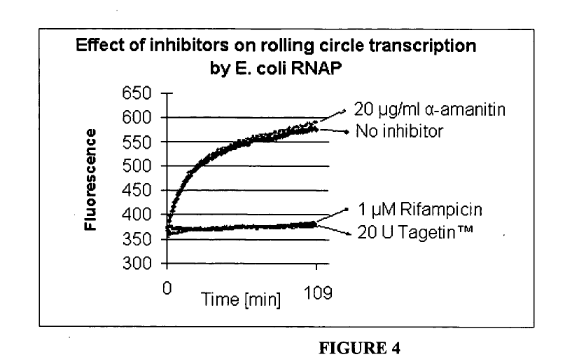

The results indicated that RNA was synthesized by E. coli RNA Polymerase Core Enzyme in the absence of rifampicin, or in the presence of α-amanitin, a selective inhibitor of eukaryotic RNA Polymerase II, whereas no RNA was synthesized in the presence of rifampicin or TAGETIN (FIG. 4). These results show that the methods of the present invention can be used to detect RNA polymerase inhibitors that are specific to prokaryotic RNA polymerases. Also, these results illustrate that the methods of the present invention allow polymerization in the presence of a fluorescent dye thus enabling detection of polymerization products in real-time.

### Example 5

### Inhibition of Prokaryotic and Eukaryotic RNA Polymerases

A single-stranded circularized 38mer DNA molecule having the same sequence as the circular 38mer used in Example 2 (Sequence I.D. No. 2) was obtained.

A reaction mixture was prepared comprising 5 pmoles of the circularized 38mer DNA molecule, 1 unit of E. coli RNA Polymerase Holoenzyme (EPICENTRE) or of soy germ RNA polymerase II, 0.5 mM of each NTP and a reaction buffer. The reaction mixture was incubated at 37° C. for 2 hours in the presence or absence of rifampicin or α-amanitin. The reaction products were visualized by agarose gel electrophoresis.

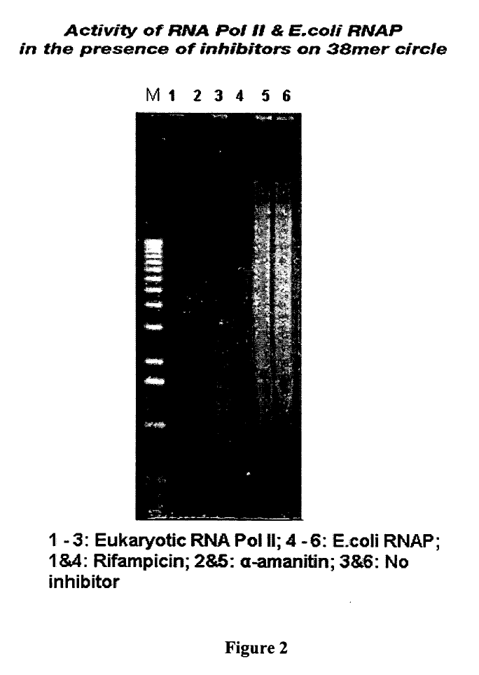

The results show that rifampicin inhibited the activity of E. coli RNA Polymerase Holoenzyme, a prokaryotic RNA polymerase, but did not have an inhibitory effect on RNA polymerase II, a eukaryotic RNA polymerase (FIG. 2). Inhibitory effects of α-amanitin on eukaryotic RNA polymerase II were observed, whereas no inhibition of the prokaryotic E. coli RNA Polymerase Holoenzyme was observed. These results indicate that the methods of the present invention can be used to identify inhibitors that specifically inhibit RNA polymerases of prokaryotes including pathogenic prokaryotes while having no inhibitory effect on eukaryotic RNA polymerases and therefore represent potential drugs against prokaryotic pathogens.

### Example 6

### The Same Circular Single-Stranded DNA Molecule Functions as Template for Multiple Different RNA Polymerases

The same single-stranded circularized 45mer DNA molecule as used in Example 1 was obtained (Sequence I.D. No. 1).

A reaction mixture was prepared containing 50 ng (about 3.6 pmoles) of the single-stranded circularized 45-mer DNA molecule, 0.5 mM of each NTP, a reaction buffer and one of the following RNA polymerases: 1 ug of E. Coli RNA polymerase Holoenzyme (EPICENTRE), 1 ug of E. coli RNA polymerase Core Enzyme (EPICENTRE), 1 ug of S. aureus RNA polymerase, 1 ug of T. thermophilus RNA polymerase, 0.25 ug of T7 RNA polymerase, 0.25 ug of T3 RNA polymerase, 0.25 ug of SP6 RNA polymerase, and 0.25 ug of N 4 mini-v RNA polymerase. The reaction mixture was incubated for 1 hour at 37° C. except for the reaction mixture comprising T. thermophilus RNA polymerase, which was incubated at 60° C. The reaction products were visualized on a 1% TAE-agarose gel.

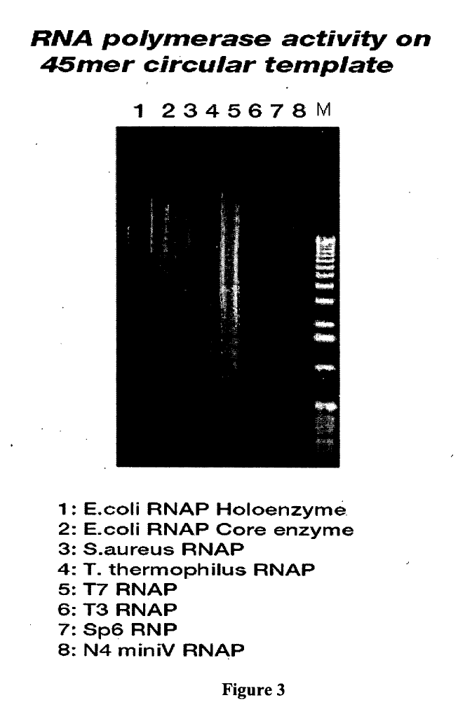

All reactions with each RNA polymerase resulted in the synthesis of RNA (see FIG. 3). This result indicated that the same single-stranded DNA circle can be used as a transcription template for a broad range of different RNA polymerases. Also, this finding illustrates that no RNA polymerase promoter sequence is needed for transcription, which is a preferred aspect of the present invention.

### Example 7

### Monitoring Rolling Circle Transcription with a Molecular Beacon

A single-stranded circularized 81 mer DNA molecule having the same sequence as the circular 81 mer used in Example 3 (Sequence I.D. No. 3) was obtained. Also, a molecular beacon was obtained having the following sequence:

(Sequence I.D. No. 4)

TET-CGCUUUUUUUUUUUUUUUU GCG dabcyl.

A reaction mixture was prepared containing 1 U of E. coli RNA Polymerase Core Enzyme, reaction buffer, 100 ng of the molecular beacon, and 0.8 pmole of the circularized 81 mer. A reaction was started by the addition of a mixture of ATP, CTP, GTP and UTP such that the concentration of each NTP was 0.5 mM in the final reaction mixture; no NTP was added to a negative control sample. Fluorescence was measured for about 3 hours in an iCycler iQ real-time PCR detection system (Bio-Rad Laboratories) using 490 nm excitation and 530 nm emission wavelengths.

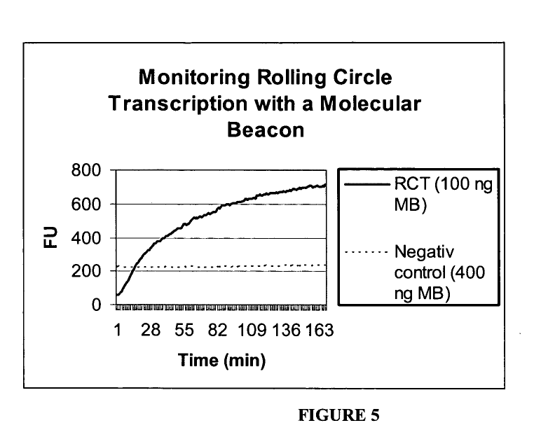

RNA production by rolling circle transcription caused an increase of the fluorescence of the Molecular beacon (FIG. 5). These results illustrate that RNA production by rolling circle transcription can be detected by Molecular Beacons.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for identifying an inhibitor of a nucleic acid polymerase activity, comprising:
a) providing
i) a single-stranded circular oligonucleotide template;
ii) a nucleic acid polymerase; and
iii) a plurality of nucleoside triphosphates;
b) incubating said template, said nucleic acid polymerase, and said nucleoside triphosphates in the presence and absence of a candidate inhibitor.

2. The method of claim 1, wherein said candidate inhibitor inhibits transcription by said nucleic acid polymerase.

3. The method of claim 1, further comprising measuring the presence or absence of a polymerization product formed in the presence and absence of said candidate inhibitor.

4. The method of claim 3, further comprising the step of comparing an amount of the polymerization product formed in the presence and absence of said candidate inhibitor; wherein a decrease in the amount of said polymerization product formed in the presence of the candidate inhibitor compared to the amount of the polymerization product formed in the absence of the candidate inhibitor indicates that said candidate inhibitor is an inhibitor of the nucleic acid polymerase activity.

5. The method of claim 1, wherein said method is performed in the absence of a primer.

6. The method of claim 1, wherein said method is performed in the presence of a primer.

7. The method of claim 1, wherein the nucleotide sequence of said template is devoid of a polymerase promoter sequence.

8. The method of claim 1, wherein the nucleotide sequence of said template comprises a polymerase promoter sequence.

9. The method of claim 1, wherein said template is DNA.

10. The method of claim 1, wherein said template is RNA.

11. The method of claim 1 wherein said nucleic acid polymerase is selected from the group consisting of a DNA-dependent RNA polymerase, an RNA-dependent RNA polymerase, a primase, a DNA polymerase, and a reverse transcriptase.

12. The method of claim 11, wherein said DNA-dependent RNA polymerase is a prokaryotic RNA polymerase.

13. The method of claim 12, wherein said prokaryotic RNA polymerase is S. aureus RNA polymerase.

14. The method of claim 11, wherein said DNA-dependent RNA polymerase is a eukaryotic RNA polymerase.

15. The method of claim 11, wherein said nucleic acid polymerase is selected from the group consisting of a eukaryotic virus polymerase and a prokaryotic virus polymerase.

16. The method of claim 4, wherein said comparing the amount of the polymerization product formed in the presence and absence of said candidate inhibitor comprises measuring fluorescence generated from a dye that undergoes fluorescence enhancement upon binding to nucleic acids.

17. The method of claim 16, wherein said dye is selected from the group consisting of RIBOGREEN, SYBR Gold, SYBR Green I, and SYBR Green II.

18. The method of claim 16, wherein said fluorescence is generated in real time.

19. The method of claim 4, wherein said comparing the amount of the polymerization product formed in the presence and absence of said candidate inhibitor comprises measuring fluorescence generated from a molecular beacon.

20. A kit for identifying an inhibitor of a nucleic acid polymerase activity, comprising:
a) a single-stranded circular oligonucleotide template;
b) a nucleic acid polymerase; and
c) a reagent for detection of transcription from said template.

21. The kit of claim 20, further comprising a plurality of nucleoside triphosphates.

22. The kit of claim 20, further comprising a plurality of inhibitors of said nucleic acid polymerase.

23. The kit of claim 20, further comprising a primer complementary to said template.

24. The kit of claim 20, wherein the nucleotide sequence of said template is devoid of a polymerase promoter sequence.

25. The kit of claim 20, wherein the nucleotide sequence of said template comprises a polymerase promoter sequence.

26. The kit of claim 20, wherein said template is DNA.

27. The kit of claim 20, wherein said template is RNA.

28. The kit of claim 20, wherein said nucleic acid polymerase is selected from the group consisting of a DNA-dependent RNA polymerase, an RNA-dependent RNA polymerase, a primase, a DNA polymerase, and a reverse transcriptase.

29. The kit of claim 28, wherein said DNA-dependent RNA polymerase is a prokaryotic RNA polymerase.

30. The kit of claim 29, wherein said prokaryotic RNA polymerase is S. aureus RNA polymerase.

31. The kit of claim 28, wherein said DNA-dependent RNA polymerase is a eukaryotic RNA polymerase.

32. The kit of claim 20, wherein said nucleic acid polymerase is selected from the group consisting of a eukaryotic virus polymerase and a prokaryotic virus polymerase.

33. The kit of claim 20, wherein said reagent comprises a dye that undergoes fluorescence enhancement upon binding to nucleic acids.

34. The kit of claim 33, wherein said dye is selected from the group consisting of RIBOGREEN, SYBR Gold, SYBR Green I, and SYBER Green II.

35. The kit of claim 20, wherein said reagent comprises a molecular beacon.

36. A method for detecting RNA polymerase activity in a sample suspected of containing an RNA polymerase, comprising:
a) providing
i) the sample suspected of containing an RNA polymerase;
ii) a single-stranded circular oligonucleotide DNA template; and
iii) a plurality of nucleoside triphosphates;
b) incubating said DNA template, said sample and said nucleoside triphosphates under conditions such that said RNA polymerase, if present, transcribes said DNA template resulting in an RNA product.

37. The method of claim 36, further comprising the step of measuring the presence or absence of said RNA product.

38. The method of claim 36, wherein said method is performed in the absence of a primer.

39. The method of claim 36, wherein said method is performed in the presence of a primer.

40. The method of claim 36, wherein the nucleotide sequence of said template is devoid of a polymerase promoter sequence.

41. The method of claim 36, wherein the nucleotide sequence of said template comprises a polymerase promoter sequence.

42. The method of claim 37, wherein said measurement of the presence or absence of the RNA product is a real-time measurement.

43. The method of claim 37, wherein said measurement of the presence or absence of the RNA product is an end-point measurement.

44. The method of claim 37, wherein said measurement of the presence or absence of the RNA product comprises measuring fluorescence from a dye that undergoes fluorescence enhancement upon binding to nucleic acids.

45. The method of claim 44, wherein said dye is selected from the group consisting of RIBOGREEN, SYBR Gold, SYBR Green I and SYBR Green II.

46. The method of claim 37, wherein said measurement of the presence or absence of the RNA product comprises measuring fluorescence from a molecular beacon.


SEQUENCE LISTING (4 sequences)

SEQ ID NO: 1 - DNA, 45 bp, Artificial Sequence
  Synthetic
ctggaggagattttgtggtatcgattcgtctcttagaggaagcta

SEQ ID NO: 2 - DNA, 38 bp, Artificial Sequence
  Synthetic
caaaagaagcggagcttcttuttttttttttttttttt

SEQ ID NO: 3 - DNA, 81 bp, Artificial Sequence
  Synthetic
agtcctcagtccacgtggtttttttttttttttttttttttgcgctagggataacagggtaatcattgccgtctgaagagg

SEQ ID NO: 4 - DNA, 22 bp, Artificial Sequence
  Synthetic
cgcuuuuuuuuuuuuuuuugcg